(19) United States
(12) Patent Application Publication
(10) Pub. No.: US 2022/0005593 A1
(43) Pub. Date: Jan. 6, 2022

(54) SYSTEM AND METHOD OF DATA COLLECTION AND PERFORMANCE ANALYSIS IN A DENTAL OFFICE

(71) Applicant: Karen A. Ergus, Kanata (CA)

(72) Inventor: Karen A. Ergus, Kanata (CA)

(21) Appl. No.: 17/239,572

(22) Filed: Jul. 21, 2021

(30) Foreign Application Priority Data
Jul. 6, 2020 (CA) 3085911

(51) Int. Cl. G16H 40/20; G16H 10/20; G16H 80/00; G06F 40/40; G06N 20/00; G16H 20/00

(52) U.S. Cl. CPC G16H 40/20; G16H 10/20; G16H 20/00; G06F 40/40; G06N 20/00; G16H 80/00

(57) ABSTRACT

A system and method is provided enabling a novel approach to data collection and performance analysis in a dental office. Members of the staff in a dental office complete questionnaires structured around the various activities performed by these Members daily. This data is transmitted to a database in a secure cloud. With the data collected, the system offers the ability to provide real-time performance analysis, solutions regarding the obstacles identified, and monthly reports. Furthermore, a practice management consultant has the ability to provide direct feedback to Members based on their inputted data.

### FIELD OF THE INVENTION

The invention relates to a system and method of data collection for a dental office using questionnaires. In particular, this invention includes performance analysis identifying where performance can be improved, courses that can be offered, where processes can be improved and potential revenue that is lost when individual service providers render services below the standard.

### BACKGROUND OF THE INVENTION

Existing dental practice management computerized systems collect data that will report on the overall practice performance of a dental practice but do not provide reports on an individual's performance at a micro level as it pertains to their day to day obstacles and duties. Presently, data that is collected is done so as a legal and professional requirement for patient care and to submit dental insurance claims.

Reports can be generated to show day-to-day production of an office. Most dental computerized systems are generally concerned about keeping track of the overall production of dental practices but not at all about the relationship between times lost when delivering services and how it can negatively affect revenues and the standard of care. Furthermore, dental systems do not analyze the time lost when service providers render services and more importantly how the ineffective use of time can affect revenues. To clarify further, dental providers and offices bill clients according to the time spent in delivering dental services because most dental fees are associated to a fee guide with a time allotment to substantiate the fees charged.

To illustrate please see the following example:
If the allotted time of 30 minutes is given to clean a 6 year old child's teeth and the provider takes 45 mins, then the extra 15 minutes used over the allotted time is lost and cannot be billed for, thus revenue is lost because in effect, the provider has taken too much time for that service.

Similarly, if less time is taken than the time scheduled for delivering services, revenue is lost because the time lost cannot be billed or given to another client.

To illustrate, let us refer to the same example above:
If the allotted time of 30 minutes is given to clean a 6 year old child's teeth and the provider takes 15 mins then the scheduled time of 15 minutes not used cannot be billed for and thus revenue is lost because the scheduled time lost cannot be allotted to another client.

It is a challenge to continually motivate providers to maintain the highest levels of production, efficiency, and standard of care. Therefore, in order to address this issue, dental practice owners seek out services from Practice Management Consultants who set up systems and suggestions to improve production and efficiency. The problem with this method is that once the Practice Consultants deliver their recommendations, the systems and suggestions are difficult to sustain by the service providers because change in behaviors is required to maintain effectiveness. This is because very few systems are in place to monitor the implementation and sustainability of the recommended changes by consultants which means service providers revert to old habits. As a result, many dollars are spent on Practice Consultants and over time, the value is lost.

The only other option that dental practice owners have is to impress upon individual service providers to maintain motivation and aspire to be productive and efficient, but this is difficult without a system of accountability.

It would be advantageous to have a system that will assist service providers in achieving efficiency and accountability so that they can achieve their performance goals and have a sustainable change in behaviour.

### SUMMARY OF THE INVENTION

A system and method are provided enabling a novel approach to data collection and performance analysis in a dental office. Members of the staff in a dental office complete questionnaires structured around the various activities performed by these Members daily in the app. This data is then transmitted to a database in a secure cloud. With the data collected, the system offers the ability to provide real-time performance analysis, solutions regarding the obstacles identified, and monthly reports.

Optionally, a practice management consultant has the ability to provide direct feedback to Members in the app based on their inputted data.

This invention collects data from all Members of a dental office (defined generally as Dentists, Dental Assistants, Dental Hygienists, Treatment Coordinators, and Office Managers). Data is collected in the form of daily questionnaires and these questions are framed around time spent on patient-based services, obstacles faced by Members, classification of periodontal status, recommended treatments and services, and optional notes. Data is also collected anonymously from patients with questions framed around satisfaction of services received, what toothbrush the patient is using, and what services the patient would like to know more about. According to one embodiment of the invention, an objective is to use the collected data and present it in a meaningful way such that it can provide insight into where a Member can improve performance, how a Member can maximize the amount of time that was scheduled for clients, courses that can be offered to promote efficiency, how processes in the office can be improved, and potential revenue that is lost when individual service providers render services below the “industry standard”. This industry standard is known as the “gold standard time”, namely a defined time allotment for a service rendered by a Member of a dental office. This time allotment represents the optimal time it takes for a Member to complete a service.

According to one embodiment of the invention, each time allotment is set based on industry data and each allotment is utilized as a baseline or benchmark when evaluating the actual time spent by a Member rendering a service. This allows for the system to be able to assess areas of where a Member can improve performance, how that Member could make proper use of their time, and potential revenue that is lost. It would be useful to have a system that is staff friendly, educational and to provide service providers with standard of care information concerning services that are relevant to their specific practice industry. Traditionally, dental practices hire consultants to visit the office and assist in achieving the same objectives. However, this can be cost-prohibitive. The present system and method will be available at a fraction of the cost, thereby enabling resources to be directed to improved patient care.

### BRIEF DESCRIPTION OF THE DRAWINGS

Embodiments of the invention will now be described by reference to the following drawings in which:

FIG. 1 is a flow chart outlining the onboarding process in the system.

FIG. 2 is showing the flow control of an Administrator for approving registrations in the system.

FIG. 3 is showing the flow control of when a Member logs into the system.

FIG. 4 is a flow chart showing the elements of the most commonly accessed sections of a system and method of data collection and performance analysis in a dental office.

FIG. 5 is a diagram representing the anatomy of a questionnaire in the system.

FIG. 6 is showing the sequence of steps involved to have metrics displayed in the Dashboard.

FIG. 7 is showing the flow of a patient filling a questionnaire in the system.

FIG. 8 is a diagram showcasing the various administrative functions available within the system.

FIG. 9 is a sequence diagram outlining the flow control of a content change in the system.

FIG. 10 is the process in which an administrator generates a report in the system.

FIG. 11 is a block diagram of a hardware implementation of an embodiment of the invention.

FIG. 12 is a diagram illustrating the concept of mapping a request to a contract in the API which is then associated to schema in the system database.

FIG. 13 is a diagram showing the authentication sequence of the system and method.

FIG. 14 is a block diagram representing a Deep Learning/Artificial Intelligence unit as part of the Data Processing Module within the system.

FIG. 15 is a sequence diagram showing the flow of communication within the system when querying for data.

### DETAILED DESCRIPTION

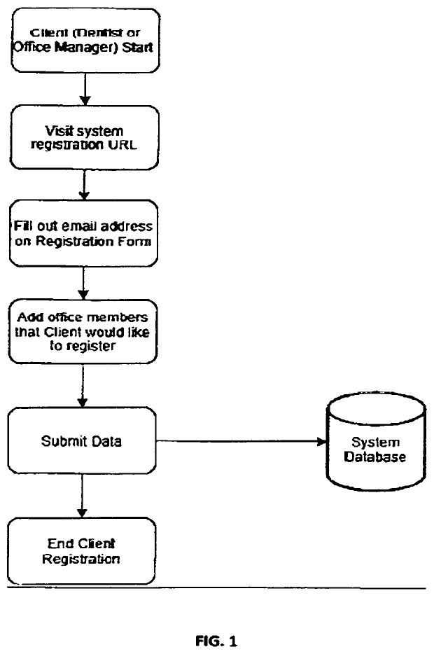

Referring to FIG. 1, there is shown a flow chart illustrating the steps taken by Dentists or Office Managers (the “client”) to onboard the system i.e. to begin to use the system. To get started with the system, a Dentist or Office Manager visits the system URL and must complete the onboarding flow:
1) Create an account using an email address
2) Add the Members they would like to register for their office—i.e. their Dental Assistant, Dental Hygienist, Treatment Coordinator, etc.

Upon completion of those steps, the registration requests are submitted to the system database where they are marked in a pending status.

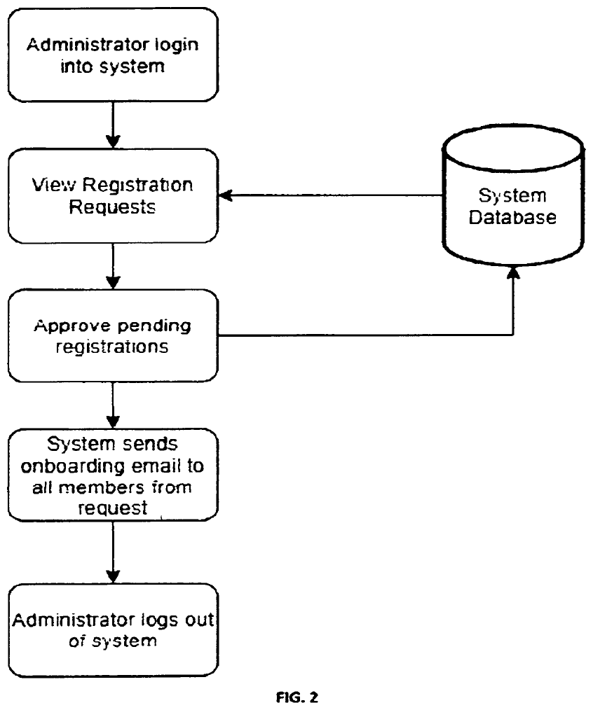

As shown in FIG. 2, an Administrator would login, view the registration requests and approve them. After approval, all Members will receive a welcome email with a set of credentials and instructions to access the system.

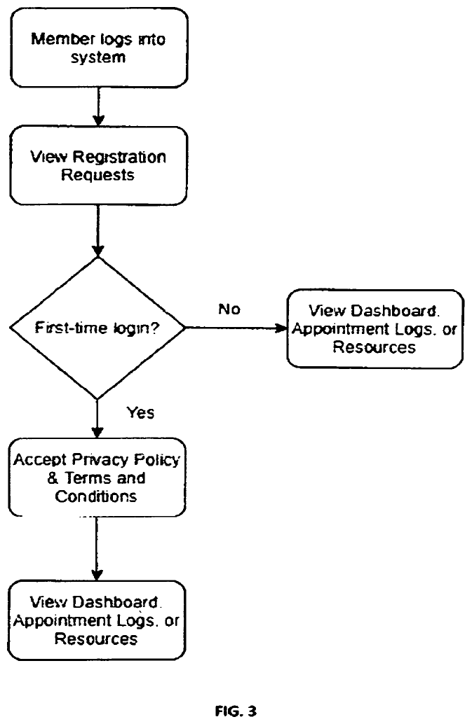

As shown in FIG. 3, there is shown the flow control of when a Member logs into the system. When a Member logs in for the first time, they must read and agree to the privacy policy and terms and conditions of the system. After agreeing to the terms, the Member is presented with a menu that contains three sections—Dashboard, Appointment Logs, and Resources. Returning Members are led directly to the Dashboard.

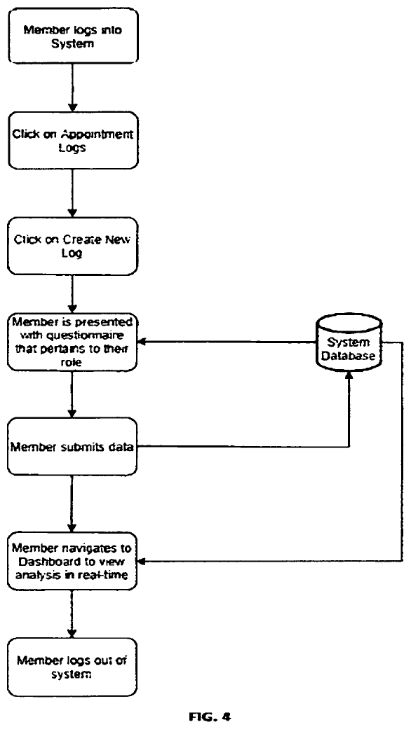

As shown in FIG. 4, a flow chart shows the elements of the most commonly accessed section of a system and method of data collection and performance analysis in a dental office. By default, the Member is directed to the Dashboard section which presents statistics that will update in real-time as the Member inputs data daily. The Appointment Logs section is the most accessed section as it is where a Member will input their data. The flow illustrated in FIG. 4 is shared for all Members of the application. A Dental Hygienist, Dental Assistant, Treatment Coordinator, Office Manager, Receptionist and Dentist will complete the flow shown in FIG. 4 daily.

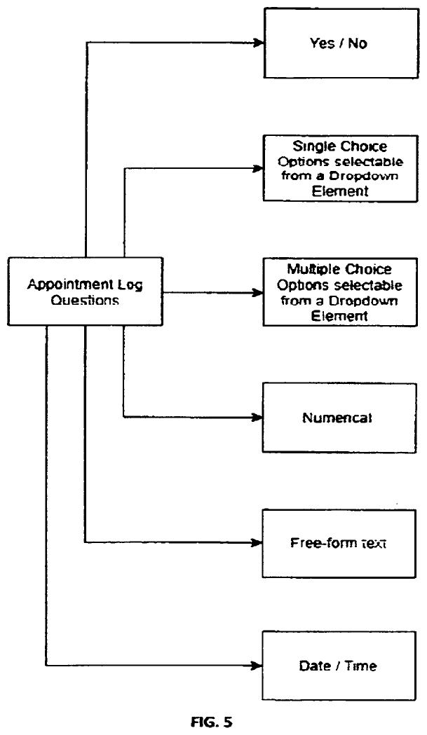

As shown in FIG. 5, each Member inputs data in the form of an Appointment Log. An appointment log is a set of questions framed around the daily tasks of a Member. The Members will have a set of questions that need to be answered. Shown in FIG. 5 is the anatomy of an appointment log, i.e. the types of questions present in a log.

The appointment log for a Dental Hygienist will capture the following information:
Patient Alias
Scheduled Appointment Time
Periodontal Type
   Shortcuts to learning resources
Obstacles Faced
Services performed along with actual time spent for each service
Other services discussed with patient
Recommended Toothbrush
Recommended Appointment Interval
Notes

It will be understood that the above questions are not an exhaustive list and may be customized to meet the dental practice's goals and objectives.

The appointment log for a Dentist will capture the following information:
Was your day stressful? If yes, please explain.
Did you get a lunch break?
Did you complete your schedule on time?
Are you happy with your production and procedures you completed today?
Did you have any issues with any of the procedures today? If yes, which one?
Where there any staff issues that you felt needed to be addressed? If yes, what were they?

It will be understood that the above questions are not an exhaustive list and may be customized to meet the dental practice's goals and objectives.

The appointment log for a Dental Assistant will capture the following information:
How many clients were seen today?
How many cancellations occurred today?
What services did you discuss with patients today?
How often did you run late today?
Did you come into work feeling sick today?
Do you feel supported by other staff Members today?
Did you help out other staff Members today?
Notes

It will be understood that the above questions are not an exhaustive list and may be customized to meet the dental practice's goals and objectives.

The appointment log for a Treatment Coordinator will capture the following information:
How many open units before the day started?
How many calls did you make today for outstanding treatment?
How many appointments booked today for another day?
How many patients asked questions about why they need the treatment?
How many patients are concerned about insurance coverage?
How many financial arrangements made today?
How many follow-up letters were sent to patients?

It will be understood that the above questions are not an exhaustive list and may be customized to meet the dental practice's goals and objectives.

The appointment log for a Receptionist captures the following information:
How many patients did not confirm today?
How many last-minute cancellations were there today?
How many last-minute appointments did you schedule today?
How many open units at the end of the day?
How many care calls were made for various appointment types?
Anyone left without paying for services rendered?
How many patients left without booking their next hygiene or restorative treatment?
How many emergencies booked today?
How many calls from individuals shopping for services?

It will be understood that the above questions are not an exhaustive list and may be customized to meet the dental practice's goals and objectives.

Lastly, the appointment log for an Office Manager captures the following information:
Any problems areas?
All goals have been met?
How many New Patients today?
How many difficult patients?
Any staff issues

It will be understood that the above questions are not an exhaustive list and may be customized to meet the dental practice's goals and objectives.

Once a Member clicks “Submit”, the response data is sent to a system backend service, where it is captured and stored in the system database. The Member then proceeds to click on Dashboard, where a set of SQL database queries are executed against the submitted data to extract the following metrics:
Top obstacles faced
Gold Standard Time vs Actual Time Spent
Overall performance score (based on actual time spent vs gold standard time)
Top recommended toothbrushes
Other services talked about the most

It will be understood that the above metrics are not an exhaustive list and may be customized to meet the dental practice's goals and objectives.

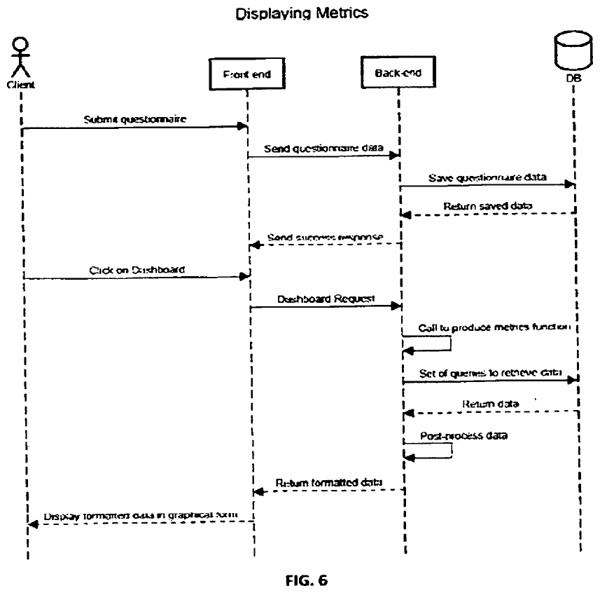

FIG. 6 shows the sequence of steps involved to have metrics displayed in the Dashboard. These metrics are presented to the Member in a graphical form each time they visit the Dashboard section of the system. The metrics update continuously based on the daily input data. Visual representation of these metrics benefits the Member as they are instantly given a snapshot of their individual performance without having to decipher a full report. Performance reports are also emailed to Members or distributed to the dental office each month.

The final section available to a Member is a section called Resources. The Resources section contains learning resources such as videos and documents that pertain to Dentistry, improving performance and efficiency.

The Sequence Diagram depicts interaction between objects in a sequential order, i.e. the order in which these interactions take place. It describes how and in what order the objects in a system function.

The call to produce metrics function and post-process data functions are occurring in the back-end portion of the system and not anywhere else. For this reason, their arrows are pointing to themselves to indicate where this action is happening.

In the system design, the front-end is the presentation layer. It represents where all the end-user interaction occurs and it is where all the browser-supported code of the web pages stored (HTML, CSS, and JavaScript). It is the part of the system responsible for receiving inputs from the end-user and presenting the application's output back to the end-user in the form of a graphical user interface (GUI). When a user clicks on a button, fills in a form, refreshes the page—These are all interactions on the front-end. The back-end component can be considered as the layer responsible for the business logic of the system. Its function is the core work performed by the application that is distinctive to it. It encompasses the functionality of the present invention that makes it uniquely valuable to its end-users. Furthermore, it is also responsible for the processing required to access and retrieve data from the database (DB). The database represents where all data is stored in the system.

As shown in the diagram, when data is being transmitted from component to component, it is in the form of a request or a response. Each arrow with a direction pointed to the right, represents a request. This is one component telling the component it's pointing to, that it is requesting some form of data. Each arrow with a direction pointed to the left, represents a response. This is a component responding back with data (in this case successfully but could respond back with error) to the component that originally requested the data.

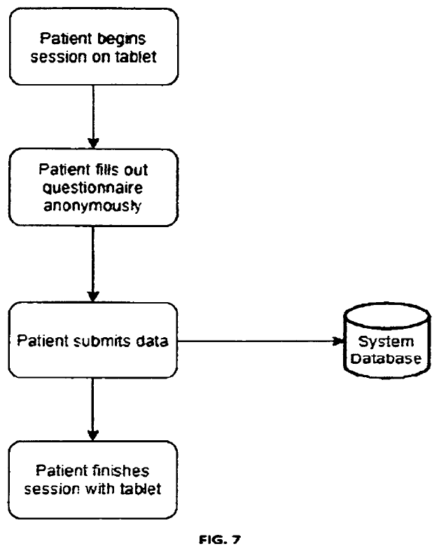

As shown in FIG. 7, not only can data be captured from staff of a dental office, the system also has the ability to capture data from the patients of a dental office. Once a patient has completed an appointment, that patient will answer a questionnaire anonymously from a tablet.

Examples of data captured from patients include the following:
Satisfaction with the services rendered (On a scale 1-10)
What toothbrush a patient is using—Oral-B, Philips or Manual
Treatment a patient would like to know more about
   Whitening
   Implants
   Fluoride Benefits
   Gingival Irrigation
   In-lays/On-lays
An input for any notes or comments

It will be understood that the above questions are not an exhaustive list and may be customized to meet the dental practice's goals and objectives.

This data will help measure the quality of care within a practice and help understand which educational topics should be an area of focus.

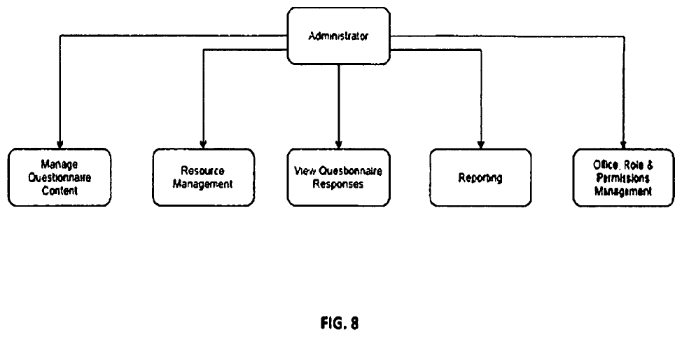

When an Administrator is using the system, they are presented with options to manage office Members, dental offices, account settings, reports, and content management. The administrative sections of the system are shown in FIG. 8 where the Administrator can:
Add/edit content:
   Add/edit other services talked about
   Add/edit questions
   Add/edit service guide entries
   Add/edit obstacles
Add/edit resources
View submitted log entries
Generate Reports
Add/manage users
Add/manage offices
Add/manage Permissions

It will be understood that the above options are not an exhaustive list and may be customized to meet the dental practice's goals and objectives.

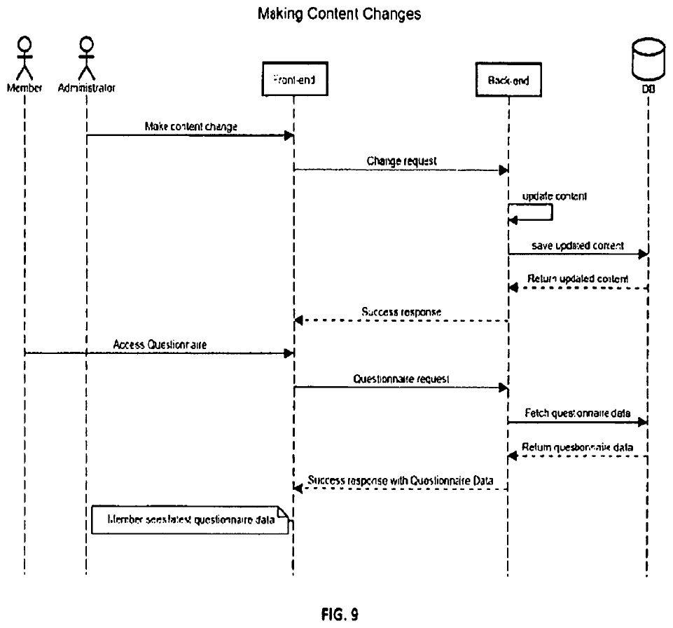

When changes in any of the content-related sections are made, those changes are reflected instantly for other Members in their questionnaires, as shown in FIG. 9. This modular design allows for new content to be reflected instantly and to avoid system downtime for making changes.

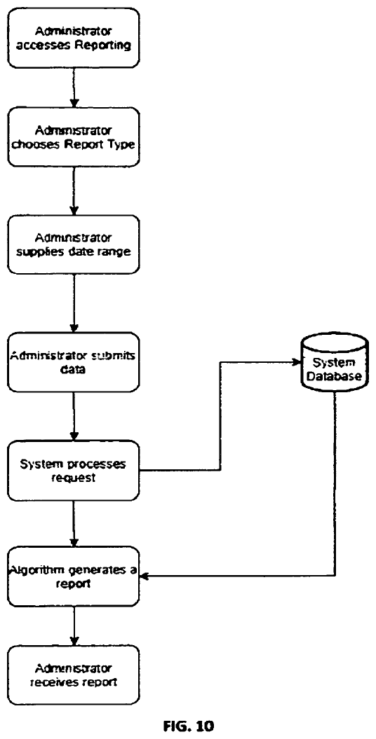

As shown in FIG. 10, the Reporting section is where an Administrator can access reports but also to input feedback such that the Office Manager/Dentist or Member will see in their monthly report. An Administrator also has a Dashboard of metrics within the purview of all offices.

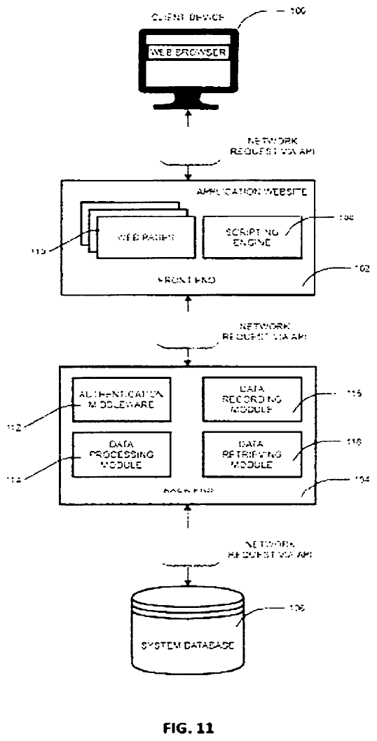

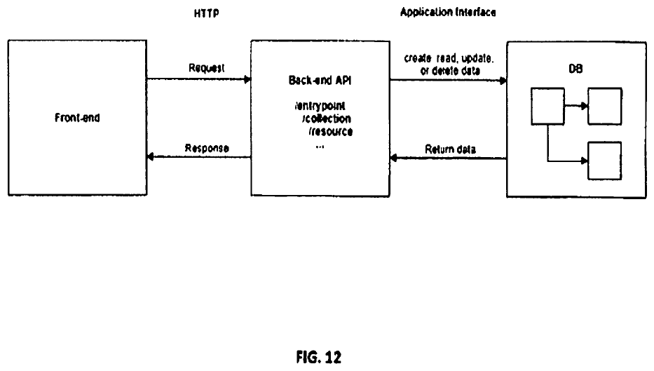

FIG. 11 represents the general architecture of a system that operates in accordance with the principles of the present invention. According to one embodiment of the invention there is provided a web-based application running on a server accessible by a client device 100, which includes any number of web-enabled devices (such as a computer or tablet). The application is accessed by users over a network such as the Internet or an intranet. The web application generally includes a computer software application that is coded in a browser-supported programming language (such as JavaScript, combined with a browser-rendered markup language like HTML, a style sheet language like CSS for describing the presentation of the rendered markup language) and uses a web browser (such as Google Chrome, Microsoft Edge, Mozilla Firefox, or Apple Safari) to render the application executable. The system also envisions other computing arrangements for the front-end 102 and back-end 104 including processing on a single machine such as a mainframe, a collection of machines, using cloud computing, or other suitable means. The application is adapted to be processed by the back-end 104 to implement features and tools of the invention, including sending data to and receiving data from the client device 100, processing data, storing data (such as questionnaires) in a database 106 as part of the server or in communication therewith. The front-end component 102 of the system is responsible for the presentation logic of the invention and provides a graphical user interface (GUI) for the client devices 100 to interact with. Communication between the client device 100, front-end 102 and back-end 104 over a network occurs via an application programming interface (API). A Representational State Transfer (REST) architectural style is used for its API and JavaScript Object Notation (JSON) to represent the data being transmitted and received. The system can also envision other protocols for interacting with the API such as gRPC, SOAP or GraphQL and other data formats such as XML or YAML. The API defines a contract of the data that it expects the front-end component 102 to pass to the back-end 104 as a request and also what the back-end 104 returns to the front-end 102 as a response. These contracts are closely tied to the schema defined in the back-end component 104. The schema is a blueprint for how data is stored and represented in the system database 106. This entire concept is best described in FIG. 12.

Each component in the overall system architecture is leveraging an open-source technology. Traditionally, systems consist of a front-end 102 and back-end 104 as a singular unit, which is best described as a monolithic architecture. However, the decision to use a split-stack architecture approach for the embodiment of the invention was meant to keep the codebase modular. It allows for simultaneous development of both the front-end 102 and back-end 104, keeps their deployments independent from one another and allows for separation of concerns. The system can envision being broken up further into smaller micro services as the scale of the application increases over time.

A. Client Device

The client device 100 may be any type of computing device that allows the end-user (the client) to interactively browse Web sites via a Web browser over a network such as the Internet or intranet. For example, the client device 100 may be a personal computer (PC) running on a “Windows™” operating system and utilizing the Microsoft Edge™ Web browser. Both Windows™ and Microsoft Edge™ are commercially available software from Microsoft Corporation. Other operating systems and Web browsers may also be used.

B. Front-End Component

The front-end component 102 can be considered as the presentation layer of the system. It represents where all the end-user interaction occurs and it is where all the browser-supported code of the web pages 110 is stored (HTML, CSS, and JavaScript). It is the part of the system responsible for receiving inputs from the end-user and presenting the application's output back to the end-user in the form of a graphical user interface (GUI). When a client device 100 accesses the application website in a web browser, a request over a network will be sent to the front-end component 102. The web server that resides in front-end component 102, will service that request via its scripting engine 108, where it is parsed and executed. The front-end component 102 will then make a request to the back-end component 104 through an API over the network to apply any necessary business logic and to retrieve data. The back-end component 104 will fetch any data required from the database 106 over the network. Once the data has been processed by the back-end component 104, it will be sent back to the front-end component 102. The front-end component 102 will then compile data in HTML format and send it to the client device 100. The client device's 100 web browser renders the HTML and presents the end-user with a graphical representation of the code. All these actions happen within a matter of seconds and are transparent to the end-user.

C. Back-End Component

The back-end component 104 can be considered as the layer responsible for the business logic of the system. Its function is the core work performed by the application that is distinctive to it. It encompasses the functionality of the present invention that makes it uniquely valuable to its end-users. Furthermore, it is also responsible for the processing required to access and retrieve data from the database 106. The system can consist of a back-end component 104 with an authentication middleware 112 and three modules—data processing 114, data recording 116 and data retrieving 118 which are representative of the four basic functions of persistent storage (Create, Read, Update and Delete). The back-end component 104 generally is coded in a server programming language (such as JavaScript, Ruby, Go) and uses a program known as a runtime to execute the server code. The system uses JavaScript for its server programming language and Node.js as its runtime environment but can also envision other programming languages, libraries, and runtimes for its implementation.

i. Authentication Middleware

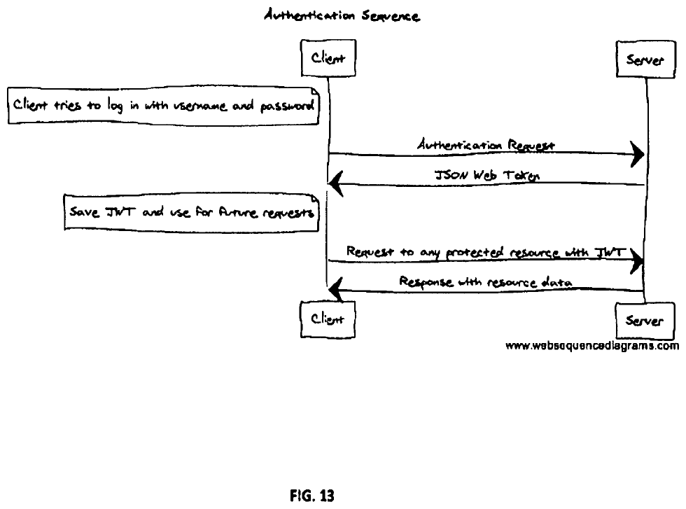

When a Member logs into the system from the client device 100, the username and password are transmitted to the back-end component 104 from the front-end component 102, through the API. The authentication middleware 112 performs a check against the database 106 to see if the credentials exist and if there is a match. If there is a match, the middleware creates a signed JSON Web Token (JWT) and returns the token to the front-end component 102 where it persisted on the client device's 100 web browser. The JWT can contain metadata about the member such as their unique identifier in the system, their role, their permissions, and the token expiration time. The system can envision additional metadata being stored in the JWT. The JWT is used to authorize a member in the system and allows the member to access any protected resources that they have permissions for. To achieve this, the JWT is appended to the request as an Authorization header. This is described in FIG. 13.

Protection of data is a serious concern and the system leverages industry best practices for security. All passwords are salted and hashed using the BCrypt password hashing function and only strong passwords are allowed in the system which must contain at least 1 letter, 1 number and a special character and be at least 8 characters in length. The system can envision stronger password requirements and additional security measures such as multi-factor authentication. Lastly, no sensitive user information such as payment information is ever stored.

ii. Data Recording Module

For data to be retrieved, it must be able to be stored. The data recording module 116 is responsible for this functionality. When a request is made from a client device 100 to create or update any data in the system, the data recording module 116 will process the request and make a connection with the database 106 to store the data. For example, when a dental hygienist inputs data in a questionnaire, the responses of each question are sent from the front-end component 102 to the back-end component 104 via API in JSON format. The data recording module 116 consumes this data, processes it, establishes a connection to the database 106, and stores the data by making a call to the database 106 using Structured Query Language (SQL).

iii. Data Processing and Data Retrieving Modules

The system stores responses for questionnaires completed by members. When a request for analysis of the stored data is made (such as a monthly performance report), it must undergo processing before it is returned to the client device 100. When a request for analysis of data is made from a client device 100, the required data is fetched from the database 106 via the data retrieving module 118. The data retrieving module 118 will perform a series of SQL queries to the database 106 to retrieve the data needed. This data is then used by the data processing module 114 to perform analysis. The retrieved data can include, but is not limited to, numerical, single choice, multiple choice, yes or no responses collected from the questionnaires. The data processing module 114 contains an algorithm that applies set of criteria to the data to help identify trends, patterns, and anomalies to help evaluate performance. For example, when evaluating the performance of a dental hygienist, one of the criteria used is to look at the actual time spent vs gold standard time. The data processing module 114 collects the actual time spent, collects the gold standard equivalent and performs a comparative analysis against the two. The compared data is then returned to the front-end component 102 which compiles the data into a graphical format such as a line graph which is rendered on the client device 100 for the end-user to see. It should be expressly understood that while a comparative analysis of gold standard time vs actual time for rendered services is discussed in connection with the present system, the invention is not strictly limited to actual time vs gold standard time comparisons. The system can be configured to evaluate data based on a different set of criteria, tailored to specific needs of a dental office.

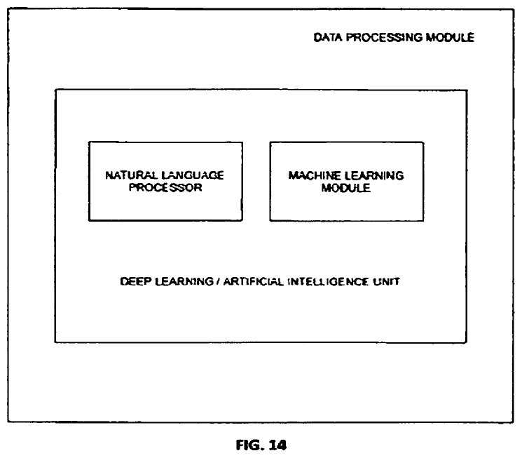

The system can envision additional analytical capabilities such as encompassing a Natural Language Processing (NLP) unit to read, decipher, understand, and make sense of the human language questionnaire responses in a manner that is valuable. The NLP unit can be used in conjunction with a Machine Learning module as a part of a larger Deep Learning/Artificial Intelligence (AI) unit that would allow the system to function as a fully self-sustained virtual consultant and to provide more detailed analysis to the end-user. This is illustrated in FIG. 14.

D. Database

Data needs to be stored somewhere for it to be retrieved. The system makes the use of a relational SQL database 106 to store and retrieve its data. Relational databases generally include a database management system such as PostgreSQL, MySQL, SQL Server or MariaDB installed on a single machine or on a collection of machines using cloud computing. The system can envision a configuration of a mix of different database systems.

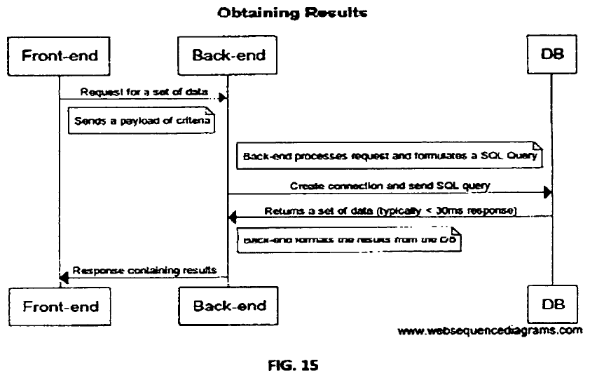

The system is designed in such a way that data can be queried easily and quickly. FIG. 15 shows the flow of communication when querying data in the system. With proper use of indexing, efficient queries can be written to retrieve data quickly.

Queries in the system, when executed, are returned in less than 30 milliseconds. Having quick response times allows us to provide an optimal user experience to be provided and avoids having the database 106 become a bottleneck.

The scope of the claims should not be limited by the preferred embodiments set forth in the examples but should be given the broadest interpretation consistent with the description as a whole.

1. A method comprising:
receiving means for receiving data from members of a dental office in response to questionnaires and receiving data from patients of a dental office in response to questionnaires by means of one or more client devices;
communication means between said client devices and a front-end component which provides a graphical user interface (GUI) for interaction with said devices and which receives said data;
communication means between said front-end component and a back-end component for storage of said data;
means for analyzing the data received based on a comparison with industry performance standards;
means for confirming the solution to improve performance standards.

2. The method according to claim 1 wherein the data processing module receives data from the data retrieving module which includes, but is not limited to, numerical, single choice, multiple choice, yes or no responses collected from the questionnaires.

3. The method according to any one of claim 1 or 2 wherein the data processing module contains an algorithm that applies set of criteria to the data to help identify trends, patterns, and anomalies to help evaluate performance in the dental office and to provide related educational resources.

4. The method according to any one of claims 1 to 4 wherein the data processing module collects the actual time spent, collects the gold standard equivalent, and performs a comparative analysis against the two.

5. The method according to any one of claims 1 to 5 wherein the data processing module wherein the compared data is then returned to the front-end component which compiles the data into a graphical format such as a line graph which is rendered on the client device for the end-user to see.

6. The method according to any one of claims 1 to 6 wherein the data processing module uses additional analytical capabilities such as encompassing a Natural Language Processing (NLP) unit to read, decipher, understand, and interpret the human language questionnaire.

7. The method according to any one of claims 1 to 7 wherein the Natural Language Processing (NLP) unit is used in conjunction with a Machine Learning module as a part of a larger Deep Learning/Artificial Intelligence (AI) unit that would allow the system to function as a fully self-sustained virtual consultant and to provide more detailed analysis to the end-user.

8. A computer-based system, comprising:
one or more client devices, for use by members of a dental office to submit data generated in response to questionnaires;
a front-end component which provides a graphical user interface (GUI) for interaction with said client devices and which receives said data;
a back-end component which interacts with said front-end component and processes said data and stores said data in a database on a server.

9. The system according to claim 8 wherein the client device is any type of computing device such as a personal computer, tablet or smartphone.

10. The system according to any one of claim 8 or 9 wherein the front-end component makes a request to the back-end component through an Application Programming Interface (API) and receives data.

11. The system according to any one of claims 8 to 10 wherein the back-end component includes authentication middleware, data processing module, data recording module and data retrieving module.

12. The system according to any one of claims 8 to 11 wherein the data processing module receives data from the data retrieving module which includes, but is not limited to, numerical, single choice, multiple choice, yes or no responses collected from the questionnaires.

13. The system according to any one of claims 8 to 12 wherein the data processing module contains an algorithm that applies set of criteria to the data to help identify trends, patterns, and anomalies to help evaluate performance in the dental office and to provide related educational resources.

14. The system according to any one of claims 8 to 13 wherein the data processing module collects the actual time spent, collects the gold standard equivalent, and performs a comparative analysis against the two.

15. The system according to any one of claims 8 to 14 wherein the data processing module wherein the compared data is then returned to the front-end component which compiles the data into a graphical format such as a line graph which is rendered on the client device for the end-user to see.

16. The system according to any one of claims 8 to 15 wherein the data processing module uses additional analytical capabilities such as encompassing a Natural Language Processing (NLP) unit to read, decipher, understand, and interpret the human language questionnaire.

17. The system according to any one of claims 8 to 16 wherein the Natural Language Processing (NLP) unit is used in conjunction with a Machine Learning module as a part of a larger Deep Learning/Artificial Intelligence (AI) unit that would allow the system to function as a fully self-sustained virtual consultant and to provide more detailed analysis to the end-user.
